(19) United States
(12) Patent Application Publication
(10) Pub. No.: US 2026/0337332 A1
(43) Pub. Date: Oct. 29, 2026

(54) PROCESS FOR MANUFACTURING A LACTIDE-CONTAINING COMPOSITION

(71) Applicant: Purac Biochem B.V., Gorinchem (NL)

(72) Inventors: Pornchai SAELIM, Gorinchem (NL); Rens VENEMAN, Gorinchem (NL); Gerrit GOBIUS DU SART, Gorinchem (NL); Chakrapong BOONMEVISES, Gorinchem (NL)

(21) Appl. No.: 19/681,723

(22) Filed: May 19, 2026

Related U.S. Application Data
(63) Continuation of application No. PCT/EP2024/082934, filed on Nov. 20, 2024.

(30) Foreign Application Priority Data
Nov. 21, 2023 (EP) 23211236.7

(51) Int. Cl. C08G 63/08; C07D 319/12; C08G 63/664

(52) U.S. Cl. CPC C08G 63/08; C07D 319/12; C08G 63/664

(57) ABSTRACT

The present disclosure relates to a process for manufacturing a lactide-containing composition, to a process for producing a polymer, to a particular lactide-containing composition, and to a use of 2,3-butanediol as an inhibitor of acetaldehyde formation in a process of manufacturing lactide. Particularly, the present disclosure relates to a process for manufacturing a lactide-containing composition comprising the steps of: providing co-oligomers derived from lactic acid and from 0.05 to 5.0 wt. % of 2,3-butanediol, and depolymerising the composition to form a lactide-containing composition.

### CROSS REFERENCE TO RELATED APPLICATIONS

The present application is a continuation of International Application No. PCT/EP2024/082934, filed Nov. 20, 2024, which claims priority to European Patent Application No. 23211236.7, filed Nov. 21, 2023, all of which are hereby incorporated by reference herein in their entireties.

### TECHNICAL FIELD OF THE INVENTION

The present disclosure relates to a process for manufacturing a lactide-containing composition, a process for producing a polymer, particular compositions comprising 2,3-butanediol, and a use of 2,3-butanediol as an inhibitor of aldehyde formation in a process of manufacturing lactide.

### BACKGROUND

Polylactide, also known as poly(lactic acid) or PLA, is a polymer that finds application in a variety of fields, ranging from packaging to disposable tableware. Polylactide is a polymer derived from lactic acid. Lactic acid is a chiral molecule and so exists as either (S)- or (R)-lactic acid. Commercially available polylactides generally contain a large proportion of(S)-lactic acid units and are usually obtained by ring-opening polymerisation of (predominantly) L-lactide, a dimer of (S)-lactic acid.

Lactides are commonly synthesised by oligomerizing lactic acid to form lactic acid oligomers via polycondensation reactions. These oligomers are then depolymerised to form a crude lactide comprising L-lactide ((S,S)-lactide), D-lactide ((R,R)-lactide), and meso-lactide ((S,R)-lactide) in presence of a catalyst. Reference is made to, for example, U.S. Pat. Nos. 5,357,035, 5,521,278, WO 2010/105143, and US 2014/031566. When the oligomers are synthesised from(S)-lactic acid, the resulting oligomer will contain mostly(S)-lactic acid units. Depolymerisation of such an oligomer will, in turn, lead to a crude lactide, wherein L-lactide is the predominant stereoisomer.

Improvements of lactide syntheses are highly sought-after. One approach to improving lactide syntheses suggested in the literature is to perform the lactide synthesis in the presence of a performance-enhancing compound. The performance-enhancing compounds described in the literature are generally high-boiling-point alcohols (more specifically, triols). For example, Wang et al. (Ind. Eng. Chem. Res., 2018, 57, 7711-7716) suggest using pentaerythritol. JP H06287278, JP H07309862, and U.S. Pat. No. 8,981,124 also describe using a high-boiling-point alcohol in a lactide synthesis.

Although the use of a high-boiling-point alcohol can improve certain aspects of a lactide synthesis, the use of a high-boiling-point alcohol is not without disadvantages. An important disadvantage is that the high-boiling-point alcohols described in the literature are not endogenous alcohols in a lactide synthesis reactor. That is, the high-boiling-point alcohols need to be added separately, because they are not normally present in a lactide synthesis. Consequently, their use brings about additional operational requirements and costs.

Thus, there is still a need in the art for an improved process for manufacturing a lactide-containing composition. The present disclosure provides such a process.

### SUMMARY OF THE INVENTION

In an aspect, the present disclosure relates to a process for manufacturing a lactide-containing composition comprising the steps of:
providing co-oligomers derived from lactic acid and from 0.05 to 5.0 wt. % of 2,3-butanediol (calculated on the total weight of the co-oligomers, as determined by gas chromatography (GC)), and
depolymerising the co-oligomers to form a lactide-containing composition.

The process according to the present disclosure requires the presence of co-oligomers derived from lactic acid and from 0.05 to 5.0 wt. % of 2,3-butanediol in the depolymerisation step of a lactide synthesis. Surprisingly, the use of such co-oligomers in the depolymerisation step was found to reduce the formation of acetaldehyde. This is advantageous, as acetaldehyde formation is indicative of degradation and reduces the overall yield of the lactide synthesis. In addition, it was surprisingly found that the presence of 2,3-butanediol units in the co-oligomers subjected to the depolymerisation step led to improved reaction kinetics (in particular, an increased net crude production rate), a decreased racemisation of lactide, and a decreased viscosity of the reactor mixture in the lactide synthesis reactor.

In another aspect, the present disclosure relates to a composition comprising co-oligomers derived from lactic acid and from 0.05 to 5.0 wt. % of 2,3-butanediol (calculated on the total weight of the co-oligomers, as determined by GC).

In another aspect, the present disclosure relates to a lactic acid-containing composition comprising lactic acid and 0.05 to 12.0 wt. % of 2,3-butanediol (calculated on the total weight of the composition).

The present inventors found that such a lactic acid-containing composition could be obtained by recycling (part of) the bottom of a lactide synthesis reactor and used to form the co-oligomers derived from lactic acid and from 2,3-butanediol as used in the process according to the disclosure, thereby eliminating the need to separately add performance-enhancing compounds, such as pentaerythritol, during a lactide synthesis.

In another aspect, the present disclosure relates to a process for producing a polylactide comprising the steps of:
manufacturing the lactide-containing composition in accordance with the process according to the disclosure, and
polymerizing at least a portion of the lactide-containing composition to form a polymer.

In another aspect, the present disclosure relates to a use of 2,3-butanediol as an inhibitor of acetaldehyde formation in a process of manufacturing lactide.

### BRIEF DESCRIPTION OF THE DRAWINGS

FIG. 1 shows a process diagram of preparing a lactide-containing composition according to a regular procedure starting from a feed comprising lactic acid and trace amounts of 2,3-butanediol.

FIG. 2 shows a process diagram of preparing a lactide-containing composition according to the present disclosure starting from a feed comprising 2,3-butanediol and lactic acid.

### DETAILED DESCRIPTION

The aspects of the invention will be discussed in more detail below. Specific advantages of the process and the compositions according to the disclosure, as well as of specific embodiments thereof, will become apparent from the further specification.

### Process for Manufacturing a Lactide-Containing Composition

As mentioned above, disclosed herein is a process for manufacturing a lactide-containing composition comprising the steps of:
providing co-oligomers derived from lactic acid and from 0.05 to 5.0 wt. % of 2,3-butanediol (calculated on the total weight of the co-oligomers, as determined by GC), and
depolymerising the co-oligomers to form a lactide-containing composition.

### Co-Oligomers

The co-oligomers subjected to the depolymerisation step are derived from lactic acid and from 0.05 to 5.0 wt. % 2,3-butanediol (calculated on the total weight of the co-oligomers, as determined by GC). It was found that the amount of 2,3-butanediol in the co-oligomers should not be too high, but also not be too low. When the amount of 2,3-butanediol is too high or too low, more acetaldehyde is produced during the lactide synthesis and the viscosity of the reaction mixture in the lactide synthesis reactor is higher (which, in turn, may lead to a higher energy consumption). Accordingly, the co-oligomers may in particular comprise (i.e., be derived from lactic acid and) 2,3-butanediol in an amount of from 0.10 to 4.5 wt. % (calculated on the total weight of the co-oligomers, as determined by GC), in particular 0.12 to 4.0 wt. %, more in particular 0.15 to 3.5 wt. %, more in particular 0.20 to 3.0 wt. %, more in particular 0.25 to 2.5 wt. %, more in particular 0.30 to 1.30 wt. %, more in particular 0.42 to 1.20 wt. %, more in particular 0.45 to 1.00 wt. %.

It will be clear that the co-oligomers may further comprise 95.0 to 99.95 wt. % of lactic acid (calculated on the total weight of the co-oligomers, as determined by GC), i.e. be derived from 95.0 to 99.95 wt. % of lactic acid and from an amount of 2,3-butanediol as disclosed herein. The co-oligomers may in particular comprise 95.5 to 99.90 wt. % of lactic acid, in particular when the co-oligomers comprise 0.10 to 4.5 wt. % of 2,3-butanediol. More preferably, the co-oligomers may comprise 96.0 to 99.88 wt. % of lactic acid, in particular when the co-oligomers comprise 0.12 to 4.0 wt. % of 2,3-butanediol. More preferably, the co-oligomers comprise 96.5 to 99.85 wt. % of lactic acid, in particular when the co-oligomers comprise 0.15 to 3.5 wt. % of 2,3-butanediol. More preferably, the co-oligomers comprise 97.0 to 99.80 wt. % of lactic acid, in particular when the co-oligomers comprise 0.20 to 3.0 wt. % of 2,3-butanediol. More preferably, the co-oligomers comprise 97.5 to 99.75 wt. % of lactic acid, in particular when the co-oligomers comprise 0.25 to 2.5 wt. % of 2,3-butanediol. More preferably, the co-oligomers comprise 98.70 to 99.7 wt. % of lactic acid, in particular when the co-oligomers comprise 0.30 to 1.30 wt. % of 2,3-butanediol. The co-oligomers may comprise 98.8 to 99.58 wt. % of lactic acid and 0.42 to 1.20 wt. % of 2,3-butanediol. The co-oligomers may comprise 99.0 to 99.55 wt. % of lactic acid and 0.45 to 1.0 wt. % of 2,3-butanediol.

As is well-known to persons skilled in the art, the amounts of lactic acid and 2,3-butanediol in the co-oligomers can readily be determined using gas chromatography. Specifically, the co-co-oligomers derived from lactic acid and from 2,3-butanediol can be methanolized (i.e., broken down via transesterification with methanol) to form methyl lactate and 2,3-butanediol. The amounts of 2,3-butanediol and methyl lactate (representative of the amount of lactic acid that was present in the composition) can then be analysed using standard gas chromatography methods.

In a typical procedure, a co-oligomer sample is first completely esterified to obtain methyl esters of any (hydroxy) acid. This is done to determine the total amount of 2,3-butanediol in the sample, irrespective of the form in which it is present in the sample (as 2,3-butanediol may be present in the sample as free 2,3-butanediol, or as 2,3-butanediol esterified with lactates). Derivatization is then generally obtained using methanol and an ion exchange resin like Amberlyst® 15, while a solvent like dichloromethane may be used for more viscous samples; typically this reaction is completed in 2 h at 90 °C. After this sample derivatization, the solution can be decanted with a syringe and injected into a GC apparatus. Such apparatus may be a Thermo-GC Trace 1300 with e.g. an Agilent® DB-WAX Ultralnert, L=30 m, i.d.=0.25 mm, df=0.25 μm GC column, hydrogen carrier gas and FID detector. A gradient oven temperature is generally used (50-200 °C) and an injection temperature of 260 °C is generally applied. Hexanoic acid may be used as internal standard.

It is known to those skilled in the art that 2,3-butanediol exists in three stereoisomeric forms: 2(R),3(R)-butanediol, 2(S),3(S)-butanediol and 2(R),3(S)-butanediol. In the GC analysis specified above, two elution peaks for 2,3-butanediol therefore will be observed (one for the R,R and S,S-isomers and another peak for the R,S-isomer). The sum of these two peaks will determine the total content of 2,3-butanediol.

The co-oligomers may further comprise dilactyl ether units (the diacid formed after etherification of two lactic acid molecules). The 2,3-butanediol and the dilactyl ether units may be present in the co-oligomer in a weight ratio of 5:1 to 1:5, in particular in a weight ratio of 2:1 to 1:2, more in particular in a weight ratio of about 1:1. Surprisingly, it was found the overall performance of the lactide synthesis was higher when the weight ratio of the 2,3-butanediol and the dilactyl ether units in the co-oligomers was as defined above. For example, it was found that the net crude production rate was higher, the viscosity of the reaction mixture in the lactide synthesis reactor was lower, the racemisation of the incoming co-oligomers was lower, and the amount of acetaldehyde produced was lower (all compared to the performance of a ‘normal’ lactide synthesis).

Like 2,3-butanediol, the total amount of dilactyl ether in an co-oligomer sample may be determined using gas chromatography after derivatization of the sample into methyl esters (and dilactyl ether specifically into its dimethyl ester). The same apparatus, settings and sample derivatization methods as described for determination of the amount of 2,3-butanediol may be employed.

The co-oligomers derived from lactic acid and from 2,3-butanediol may have a degree of polymerisation of from 2 to 80, in particular from 3 to 60, more in particular from 4 to 40, more in particular from 5 to 20, more in particular between 10 and 15, as calculated from the concentration of end-groups as determined by titration.

### Oligomerisation

The co-oligomers disclosed above can be produced in accordance with procedures known to those skilled in the art. Reference can be made to e.g. U.S. Pat. Nos. 5,247,058, 5,258,488 and WO 95/09879.

Briefly, in an oligomerisation step, a low-molecular weight polylactide is formed by condensation polymerisation of lactic acid and 2,3-butanediol. The condensation generally involves subjecting lactic acid and 2,3-butanediol to sub-atmospheric pressure and an elevated temperature to induce polymerisation by removal of water. The process for manufacturing a lactide-containing composition disclosed herein may, thus, further comprise a step of oligomerising lactic acid and 2,3-butanediol to provide the co-oligomers disclosed herein.

The oligomerisation step of the processes according to the disclosure may comprise condensing lactic acid and 2,3-butanediol at a temperature of 150 to 230 °C, in particular 160 to 215 °C, more in particular 170 to 200 °C, more in particular 175 to 185 °C. The lactic acid is preferably (S)-lactic acid. The oligomerisation step may be done at a pressure of 100 to 10000 Pa, in particular 250 to 7500 Pa, more in particular 300 to 5000 Pa, more in particular 300 to 1000 Pa. Thus, the oligomerisation step may comprise condensing(S)-lactic acid and 2,3-butanediol at a temperature of 150 to 230 °C and at a pressure of 100 to 10000 Pa.

### Depolymerisation

In the process according to the disclosure, the co-oligomers are then subjected to a depolymerization step, optionally in the form of a composition comprising the co-oligomers derived from lactic acid and from 2,3-butanediol, and one or more further components. The co-oligomers are subjected to the depolymerisation step to manufacture a lactide-containing composition. The lactide-containing composition generally comprises L-lactide, D-lactide, and meso-lactide. The lactide-containing composition may comprise lactides in a total amount of more than 80 wt. %, in particular more than 90 wt. %, more in particular more than 95 wt. %, more in particular more than 97 wt. %. The lactide-containing composition may comprise 60 to 99 wt. % L-lactide, in particular 75 to 98 wt. % L-lactide, more in particular 80 to 97 wt. % L-lactide, more in particular 85 to 96 wt. % L-lactide. The balance may be D-lactide and meso-lactide.

As mentioned, the co-oligomers derived from lactic acid and from 2,3-butanediol may be subjected to the depolymerisation step as a composition comprising the co-oligomers derived from lactic acid and from 2,3-butanediol, as well as one or more further components. The composition may comprise the co-oligomers, lactic acid, and 2,3-butanediol in a total amount of from 80.0 to 99.9 wt. % (based on the total weight of the composition). It may be preferred for the composition to comprise the co-oligomers, lactic acid and 2,3-butanediol in a total amount of from 85.0 to 99.0 wt. %, in particular 90.0 to 98.5 wt. %, more in particular 95.0 to 98.0 wt. %. The one or more further components may, for example, be free lactic acid, free 2,3-butanediol, and/or free dilactyl ether.

It will be evident that a combination of the features discussed above may be present in the composition subjected to the depolymerisation step. For example, the composition subjected to the depolymerisation step may comprise the co-oligomers derived from lactic acid and from 2,3-butanediol in an amount of from 80.0 to 99.9 wt. % (calculated on the total weight of the composition), wherein the co-oligomers are derived from lactic acid and from 0.05 to 5.0 wt. % of 2,3-butanediol (calculated on the total weight of the co-oligomers, as determined by GC). Other combinations of features are also envisaged.

The depolymerisation step may be done at a temperature of 150 to 230 °C. The amounts of L-lactide, D-lactide, and meso-lactide formed in the depolymerisation step show some dependence on temperature. While the ratio of L- and D-lactide will remain constant, the amount of meso-lactide may be optimised (minimised) by lowering the temperature of synthesis. Therefore, the depolymerisation reaction is preferably carried out at a temperature of 160 to 225 °C, in particular 170 to 220 °C, more in particular 180 to 215 °C, more in particular 185 to 200 °C.

The depolymerisation step may be done at a pressure of 100 to 10000 Pa, in particular 250 to 7500 Pa, more in particular 300 to 5000 Pa, more in particular 300 to 1000 Pa.

The depolymerisation step may be done in the presence of a depolymerisation catalyst. The depolymerisation catalyst is preferably selected from the group consisting of tin(II)chloride, tin(II)bromide, tin(IV)chloride, tin(IV)bromide, tin(II)oxide, tin(II)bis(2-ethylhexanoate), butyltin tris(2-ethyl hexanoate), monobutyltin oxide, dibutyltin dilaurate, tetraphenyltin, lead(II)oxide, zinc stearate, zinc lactate, antimony triacetate, antimony (2-ethyl hexanoate), bismuth (2-ethylhexanoate), calcium stearate, and magnesium stearate, titanium tetrabutoxide and titanium isopropoxide. More preferably, the depolymerisation catalyst is selected from the group consisting of tin(II)chloride, tin(II)bromide, tin(IV)chloride, tin(IV)bromide, tin(II)oxide, tin(II)bis(2-ethylhexanoate), butyltin tris(2-ethyl hexanoate), monobutyltin oxide, dibutyltin dilaurate, and tetraphenyltin. More preferably, the depolymerisation catalyst is tin(II)bis(2-ethylhexanoate).

The depolymerisation catalyst may be present in an amount of 200 to 1800 ppm Sn, in particular 300 to 1500 ppm Sn, more in particular 400 to 1200 ppm Sn, more in particular 500 to 1100 ppm Sn. The amount of depolymerisation catalyst is determined in the bottom of the lactide synthesis reactor.

It follows from the above that the depolymerisation reaction may be carried out at a temperature of 160 to 230 °C, at a pressure of 100 to 10000 Pa, and in the presence of a depolymerisation catalyst. Suitable combinations of temperature, pressure, and depolymerisation catalyst can be derived from the above and implemented by the skilled person. In a particularly preferred embodiment, the depolymerisation reaction is carried out at a temperature of 190 to 200 °C, at a pressure of 100 to 1000 Pa, and in the presence of a depolymerisation catalyst is selected from the group consisting of tin(II)chloride, tin(II)oxide, tin(II)bromide, tin(IV)chloride, tin(IV)bromide, tin(II)oxide, tin(II)bis(2-ethylhexanoate), butyltin tris(2-ethyl hexanoate), monobutyltin oxide, dibutyltin dilaurate, and tetraphenyltin.

To increase the amount of L-lactide and D-lactide synthesised during the depolymerisation step, a racemizing agent may be added. Suitable racemizing agents include hydroxide salts (e.g., LiOH, NaOH, KOH, Mg(OH₂), and the like, preferably NaOH) and acetate salts (e.g., sodium acetate, potassium acetate). Other suitable racemizing agents are known in the art and include metal salts of alkyl alcohols (i.e., salts of the structure X—O—R, wherein X is a metal selected from the group consisting of Li, Na, and K and wherein R is a substituted or unsubstituted C1-8 alkyl, e.g., a tert-butoxide salt), pyridines (preferably 1,4-lutidine, 2,6-lutidine, 3,5-lutidine, 2,6-di-tert-butylpyridine, or 4-dimethylaminopyridine), and non-nucleophilic bases (e.g., quinuclidine, 1,4-diazabicyclo [2.2.2]octane (DABCO), 1,8-diazabicyclo [5.4.0]undec-7-ene (DBU), 1,5-diazabicyclo(4.3.0) non-5-ene (DBN), and the like). The racemizing agent may be added in an amount of from 500 to 5000 ppm, in particular 750 to 2500 ppm, more in particular 900 to 1100 ppm.

The depolymerisation step is generally performed in a reactor. The reactor may comprise from 10 to 80 vol. % (as calculated on the reactor's volume) of the co-oligomer-containing composition, in particular 15 to 60 vol. % of the co-oligomer-containing composition, more in particular 20 to 50 vol. % of the co-oligomer-containing composition. This is because operating at a volume as defined above contributes to a reduced racemization in the reaction mixture.

### Process Descriptions

To illustrate the process according to the disclosure further, some processes according to the disclosure are discussed in detail below.

In an example of a process according to the disclosure, (the composition comprising) the co-oligomers can be provided by adding 2,3-butanediol as such to ‘fresh’ lactic acid (i.e., substantially pure lactic acid (such as a composition comprising lactic acid and <0.03 wt. % of 2,3-butanediol)) and oligomerising the resulting mixture using methods known in the art. The co-oligomers so-obtained are subjected to a depolymerization step as disclosed above to manufacture the lactide-containing composition, which can optionally be processed as desired.

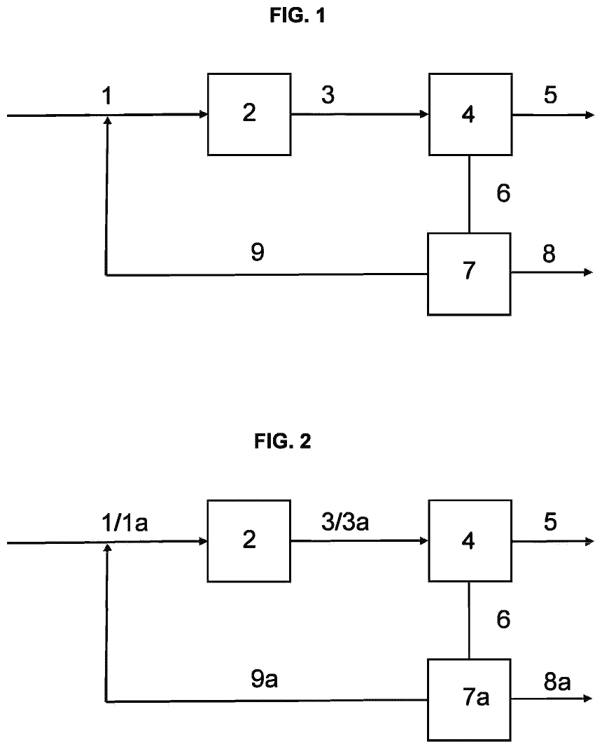

Another example of a process according to the disclosure is depicted in FIG. 2, which will be discussed below. To emphasize the differences between the process depicted in in FIG. 2 and a regular lactide synthesis, a regular lactide synthesis is depicted in FIG. 1 and described first.

FIG. 1 depicts a regular lactide synthesis. In such a synthesis, feed (1) comprising lactic acid and at most trace amounts of 2,3-butanediol is sent to an oligomerisation reactor (2), in which a condensation reaction takes place under elevated temperatures and reduced pressures (thereby allowing molecules to oligomerise and water to evaporate). The resulting composition (3) comprising lactic acid oligomers (derived from the lactic acid and from the trace amounts of 2,3-butanediol) is sent to a lactide synthesis reactor (4), in which the lactic acid oligomers are converted into a mixture comprising lactide molecules via catalysed backbiting. The resulting mixture comprising lactic acid, lactide, and 2,3-butanediol is separated (e.g. by distillation) to form a lactide stream (5) and bottom stream (6) comprising lactic acid and 2,3-butanediol. Bottom stream (6) is enriched in 2,3-butanediol as compared to the feed (1) and the composition comprising lactic acid oligomers (3). The bottom stream (6) is treated in one or more reactors (7) to separate lactic acid from contaminants. The contaminants are purged via purge stream (8). In a regular lactide synthesis, 2,3-butanediol is considered a contaminant and so purged from the system via purge stream (8). A purified lactic acid stream (9) is then recycled and combined with feed (1). Because the 2,3-butanediol is purged from the system, there is no build-up of 2,3-butanediol in the system and substantially no 2,3-butanediol is incorporated into the oligomers, even after numerous recycles.

In contrast, in an integrated process according to the disclosure (depicted in FIG. 2), bottom stream (6) is treated in one or more reactors (7a) to separate lactic acid from most contaminants (which are purged via purge stream (8a)). However, crucially, 2,3-butanediol is not (fully) removed from the lactic acid, thereby forming a composition (9a) comprising lactic acid and 2,3-butanediol (e.g. from 0.05 to 12 wt. % of 2,3-butanediol). This lactic acid-containing composition is then combined with the feed (1) to form a feed (1a) (also referred to herein as “lactic acid-containing composition”) comprising lactic acid and 0.05 to 5.0 wt. % of 2,3-butanediol. This feed (1a) is oligomerised to form the co-oligomers (3a) derived from lactic acid and from 0.05 to 5.0 wt. % of 2,3-butanediol, which are subsequently depolymerised in the depolymerisation reactor (4) to form the lactide-containing composition according to the disclosure. It should be noted that compositions comprising lactic acid and 2,3-butanediol may also be derived from other stages of a (poly)lactide manufacturing process, such as from the bottom of a distillation tower.

As yet another example, the co-oligomers can be obtained by oligomerising lactic acid to form lactic acid oligomers using methods known in the art and adding 2,3-butanediol to the lactic acid oligomers. Accordingly, in some embodiments, the step of providing the composition comprises combining lactic acid oligomers and 2,3-butanediol to form a mixture and oligomerising the mixture to provide the composition comprising co-oligomers derived from lactic acid and from 0.05 to 5.0 wt. % of 2,3-butanediol disclosed above. In these embodiments, the 2,3-butanediol is preferably added when the lactic acid oligomers are still short (e.g., when the lactic acid oligomers have a degree of polymerisation of from 2 to 10), as this results in a greater net crude production rate. The 2,3-butanediol is generally added before a depolymerisation catalyst (or ‘backbiting catalyst) is added. Suitable depolymerisation catalysts have been disclosed above.

In preferred embodiments, the process according to the disclosure is an integrated process comprising the steps of:
separating a mixture comprising lactic acid, lactide, and 2,3-butanediol in one or more steps to form a lactic acid-containing composition comprising lactic acid and 0.05 to 12.0 wt. % of 2,3-butanediol,
using at least a portion of the lactic acid-containing composition to provide the (composition comprising) co-oligomers derived from lactic acid and from 0.05 to 5.0 wt. % of 2,3-butanediol (calculated on the total weight of the co-oligomers, as determined by GC), and
depolymerising (the composition comprising) the co-oligomers to form the lactide-containing composition.

In the depolymerisation step of the integrated process, a mixture comprising lactic acid, lactide, and 2,3-butanediol is separated to form a lactic acid-containing composition comprising lactic acid and 0.05 to 12 wt. % of 2,3-butanediol. Separating the mixture can be done using methods and means known in the art. The separating may, for example, be done by means of distillation, which can remove lactide from the lactic acid and the 2,3-butanediol. The separating may be done in one or more steps.

The separation may result in the formation of a lactic acid-containing composition comprising lactic acid and 0.05 to 5.0 wt. % of 2,3-butanediol (as calculated on the total weight of the lactic acid-containing composition). The lactic acid-containing composition may preferably comprise 2,3-butanediol in an amount of from 0.15 to 4.0 wt. %, in particular 0.20 to 3.0 wt. %, more in particular 0.25 to 2.5 wt. % (as calculated on the total weight of the lactic acid-containing composition), more in particular 0.30 to 1.30 wt. % (as calculated on the total weight of the lactic acid-containing composition), more in particular 0.42 to 1.20 wt. % of 2,3-butanediol (calculated on the total weight of the lactic acid-containing composition), more in particular 0.45 wt. % to 1.0 wt. % of 2,3-butanediol (calculated on the total weight of the lactic acid-containing composition). The lactic acid-containing composition may further comprise dilactyl ether. The lactic acid-containing composition may comprise 2,3-butanediol and dilactyl ether in a weight ratio of 5:1 to 1:5, in particular in a weight ratio of 2:1 to 1:2, more in particular in a weight ratio of about 1:1. When the lactic acid-containing composition has a weight ratio as defined above, the lactic acid-containing composition may comprise 5.0 to 12 wt. % of 2,3-butanediol.

The lactic acid-containing composition disclosed above may be hydrolysed. The (optionally hydrolysed) lactic acid-containing composition may be purified. Accordingly, in some embodiments (of the integrated process), the lactic acid-containing composition comprising lactic acid and 0.05 to 12 wt. % of 2,3-butanediol is hydrolysed and purified prior to being used to provide (the composition comprising) co-oligomers derived from lactic acid and from 2,3-butanediol.

At least a portion of the lactic acid-containing composition may be used in the integrated process to provide the (composition comprising) co-oligomers derived from lactic acid and from 0.05 to 5.0 wt. % of 2,3-butanediol. This may be achieved in one or more steps. For example, the lactic acid-containing composition may be recycled to the oligomerisation reactor (in particular when the lactic acid-containing composition comprises lactic acid and 0.05 to 5.0 wt. % of 2,3-butanediol). The lactic acid-containing may also be mixed with other lactic acid streams (e.g. comprising lactic acid and less than 0.1 wt. % of 2,3-butanediol). This may be advantageous when the lactic acid-containing composition comprises lactic acid and e.g. 5.0 to 12 wt. % of 2,3-butanediol.

Accordingly, in an embodiment, the process according to the disclosure is an integrated process comprising the steps of:
separating a mixture comprising lactic acid, lactide, and 2,3-butanediol in one or more steps to form a lactic acid-containing composition comprising lactic acid and 0.05 to 12 wt. % of 2,3-butanediol,
using at least a portion of the lactic-acid containing composition to form a feed comprising 0.1 to 10.0 wt. % (in particular 1.0 to 5.0 wt. %) of the lactic-acid containing composition and 90.0 to 99.9 wt. % (in particular 95.0 to 99.0 wt. %) of one or more compositions comprising lactic acid and less than 0.1 wt. % of 2,3-butanediol,
oligomerizing the feed to provide co-oligomers derived from lactic acid and from 0.05 to 5.0 wt. % of 2,3-butanediol, and
depolymerising the co-oligomers to form a lactide-containing composition.

The integrated processes disclosed herein were found to be particularly advantageous, as they did not require separate addition of 2,3-butanediol as such. Instead, the 2,3-butanediol was enriched in the lactide synthesis reactor, recycled, and used as a performance-enhancing compound.

In some embodiments, the lactide-containing composition manufactured according to the disclosure is separated to form a lactide stream and a stream comprising 2,3-butanediol; and at least a portion of the stream comprising 2,3-butanediol is recycled in one or more steps (e.g., directly or after an optional hydrolysis step) to an oligomerisation step, wherein the oligomerisation step comprises oligomerising lactic acid to form co-oligomers derived from lactic acid and from 2,3-butanediol.

It may be desirable to purify the lactide-containing composition manufactured using the process according to the disclosure. Accordingly, the (integrated) process according to the disclosure may further comprise a step of purifying the lactide-containing composition. The step of purifying the lactide-containing composition may comprise purification by distillation, solvent crystallisation, melt crystallisation, or a combination thereof. A crystallisation approach (e.g., solvent crystallisation or melt-crystallisation) may be preferred, as crystallisation allows one to obtain a substantially pure (i.e., >99% purity) product stream comprising lactide. This is advantageous, because such a pure, racemic product stream can be sent to a polymerisation reactor to form an almost colourless polylactide. A combination of distillation and crystallisation may also be desired.

Also disclosed herein is a lactide-containing composition obtainable by the process for manufacturing a lactide-containing composition according to the disclosure.

Also disclosed herein is a use of 2,3-butanediol as an inhibitor of acetaldehyde formation in a lactide synthesis, optionally in an amount of 0.05 to 5.0 wt. % of the weight of the co-oligomers subjected to depolymerization in the lactide synthesis reactor.

### Process for Producing a Polymer

The process for manufacturing a lactide-containing composition results in the manufacture of a lactide-containing composition that is suitable for the synthesis of polymers, in particular polylactides. Therefore, disclosed herein is also a process for producing a polymer, wherein the process comprises the steps of: manufacturing a lactide-containing composition using the process according to the disclosure (see above and the claims); and polymerizing at least a portion of the lactide-containing composition to form a polymer.

The polymer may be: a polylactide; a co-polymer derived from lactic acid monomers and a monomer from a further (di) lactone selected from the group consisting of glycolide, mandelide and other substituted glycolides, caprolactone, a four- or five-membered carbonate monomer (such as trimethylene carbonate or propylene carbonate), and sugar-derived carbonates; or a ter-polymer derived from any of the monomers described above. The polymer is preferably a polylactide.

Generally, polymerisation will be carried out by providing the lactides to a polymerisation reactor, where they will be subjected to polymerisation conditions, usually in the presence of a polymerisation catalyst. Suitable polymerisation conditions are known in the art. They may, for example, include reacting the lactide at a temperature of 100 to 225 °C, in particular 120 to 220 °C, more in particular 130 to 210 °C. Suitable polymerisation catalysts are also known in the art. The catalysts described above for the oligomerisation of lactic acid and 2,3-butanediol may also be used here and are optionally used in catalytically effective amounts, e.g., 1 to 2000 ppm (calculated on the weight of the monomer). The polymerisation reaction is usually allowed to continue until the governing thermal equilibrium concentration of residual lactide is reached, typically between 3 and 8 wt. % at the temperatures mentioned. Once the desired conversion is reached, the polymerisation catalyst is often deactivated through the addition of a catalyst deactivating agent, as this stabilises the polylactide product against catalysed backbiting, allows low residual lactide levels below 0.5 wt. % and accordingly affords the final product a thermal stability guaranteeing suitability for melt processing at PLA converters. The resulting polylactide may have an absolute number average molecular weight (Mn) of from 20 to 150 kg/mol, in particular from 35 to 100 kg/mol, as determined by gel permeation chromatography using light scattering detection.

It follows the disclosure also relates to an integrated process comprising the steps of:
providing co-oligomers derived from lactic acid and from 0.05 to 5.0 wt. % of 2,3-butanediol (calculated on the total weight of the co-oligomers, as determined by gas chromatography (GC)), and
depolymerising the co-oligomers to form a lactide-containing composition,
optionally purifying the lactide-containing composition to form a purified lactide-containing composition, and
polymerizing at least a portion of the (optionally purified) lactide-containing composition to form a polylactide.

It also follows the disclosure relates to an integrated process comprising the steps of:
separating a mixture comprising lactic acid, lactide, and 2,3-butanediol in one or more steps to form a lactic acid-containing composition comprising lactic acid and 0.05 to 12.0 wt. % of 2,3-butanediol,
using at least a portion of the lactic acid-containing composition to provide (the composition comprising) the co-oligomers derived from lactic acid and from 0.05 to 5.0 wt. % of 2,3-butanediol (calculated on the total weight of the co-oligomers, as determined by GC),
depolymerising (the composition comprising) the co-oligomers to form the lactide-containing composition,
optionally purifying the lactide-containing composition to form a purified lactide-containing composition, and
polymerizing at least a portion of the (optionally purified) lactide-containing composition to form a polylactide.

It also follows the disclosure relates to an integrated process comprising the steps of:
separating the mixture in one or more steps to form a lactic acid-containing composition comprising lactic acid and 0.05 to 12.0 wt. % of 2,3-butanediol,
using at least a portion of the lactic-acid containing composition to form a feed comprising 0.1 to 10.0 wt. % (in particular 1.0 to 5.0 wt. %) of the lactic acid-containing composition and 90.0 to 99.9 wt. % (in particular 95.0 to 99.0 wt. %) of one or more compositions comprising lactic acid and less than 0.1 wt. % of 2,3-butanediol,
oligomerizing the feed to provide co-oligomers derived from lactic acid and from 0.05 to 5.0 wt. % of 2,3-butanediol, and
depolymerising the co-oligomers to form a lactide-containing composition,
optionally purifying the lactide-containing composition to form a purified lactide-containing composition, and
polymerizing at least a portion of the (optionally purified) lactide-containing composition to form a polylactide.

It will be evident that all the preferences defined for the general process above are equally applicable to these integrated processes.

Also disclosed herein is a polylactide obtainable by the process for producing a polylactide according to the disclosure.

### Lactic Acid-Containing Composition

In another aspect, the present disclosure relates to a lactic acid-containing composition comprising lactic acid and 0.05 to 12 wt. % of 2,3-butanediol (calculated on the total weight of the lactic acid-containing composition). The lactic acid-containing composition may comprise 0.05 to 5.0 wt. % of 2,3-butanediol, in particular 0.10 to 4.5 wt. % of 2,3-butanediol, in particular 0.12 to 4.0 wt. % of 2,3-butanediol, more in particular 0.15 to 3.5 wt. % of 2,3-butanediol, more in particular 0.20 to 3.0 wt. % of 2,3-butanediol, more in particular 0.25 to 2.5 wt. % of 2,3-butanediol, more in particular 0.30 to 1.3 wt. % of 2,3-butanediol, more in particular 0.42 to 1.20 wt. % of 2,3-butanediol, more in particular 0.45 to 1.0 wt. % of 2,3-butanediol. The lactic acid-containing composition may also comprise 5.0 to 12 wt. % of 2,3-butanediol and, optionally, dilactyl ether in a weight ratio of 2,3-butanediol to dilactyl ether of 5:1 to 1:5.

The lactic acid-containing composition may comprise lactic acid in a total amount of from 88.0 to 99.95 wt. % (calculated on the total weight of the lactic acid-containing composition). It is preferred the lactic acid-containing composition comprises 95.0 to 99.95 wt. % of lactic acid, in particular 95.5 to 99.90 wt. % of lactic acid, more in particular 96.0 to 99.88 wt. % of lactic acid, 96.5 to 99.85 wt. % of lactic acid, more in particular 97.0 to 99.80 wt. % of lactic acid, more in particular 97.5 to 99.75 wt. % of lactic acid, more in particular 98.7 to 99.30 wt. % of lactic acid.

As explained earlier, the present inventors found that such a lactic acid-containing composition was particularly useful in the process of the present disclosure. More specifically, the present inventors found that such a lactic acid-containing composition could be obtained from the bottom of the lactide synthesis reactor and recycled to an earlier step in the process, thereby eliminating the need to separately add performance-enhancing compounds.

Combinations of the embodiments disclosed above are envisioned as part of the disclosure. When amounts, concentrations, dimensions and other parameters are expressed in the form of a range, a preferable range, an upper limit value, a lower limit value or preferable upper and lower limit values, it should be understood that any ranges obtainable by combining any upper limit or preferable value with any lower limit or preferable value are also specifically disclosed, irrespective of whether the obtained ranges are clearly mentioned in the context. In addition, it should be understood that all percentages mentioned herein are weight percentages, unless specified otherwise.

All documents mentioned herein are incorporated by reference in their entirety or, alternatively, to provide the disclosure for which there were specifically relied upon.

### EXAMPLES

The following examples will illustrate the practice of the invention in some preferred embodiments and are not intended to be limiting. Other embodiments within the scope of the invention will be apparent to the skilled person.

### Example A: 2,3-Butanediol Enhancing Lactide Synthesis Rates and Suppressing Side Reactions

This example compares two different cases.

In the reference case (Reference), crude lactide was produced with lactic acid having typical commercial quality specifications. As shown in the Table below, the feed stream sent to the oligomerisation reactor in the Reference example contained <0.03 wt. % of 2,3-butanediol. The oligomers formed in the oligomerisation reactor were oligomers derived from lactic acid and from <0.03 wt. % of 2,3-butanediol.

In the second case (Example 1), crude lactide was produced from recycled lactic acid. The recycled lactic acid was produced (entirely) from purge streams of the lactide process. The purge streams are enriched in 2,3-butanediol, as a result of which the amount of 2,3-butanediol in Example 1's feed stream was higher than the amount of 2,3-butanediol in the Reference's feed stream. The co-oligomers formed in the oligomerisation reactor were co-oligomers derived from lactic acid and from 1.5 wt. % of 2,3-butanediol.

In both cases (i.e., the Reference example and Example 1), the feed streams were oligomerised by feeding the feed stream to a series of reactors according to procedures known to those skilled in the art. The last reactor was operated at temperatures of from 175 to 185 °C and pressures of from 300 to 500 Pa, yielding lactic acid (co-)oligomers with a high-enough degree of polymerisation for efficient lactide formation. This (co-)oligomer-containing composition was subsequently fed to a continuous lactide synthesis reactor. The lactic acid (co-)oligomers in the (co-)oligomer-containing composition were converted into crude lactide in presence of a Sn-based catalyst (940-1040 ppm Sn), at a temperature in the range of 185 to 200 °C, and a pressure of from 300 to 500 Pa. There was no significant temperature difference between the Reference Example and Example 1. A heavies stream was continuously extracted from the bottom of the lactide synthesis reactor and the composition of this heavies stream was analysed.

The table below summarises the performance of the lactide synthesis reactor for the two different cases. The second column (Reference Example) in the table shows the performance in the case where no 2,3-butanediol was detected in feed and the bottom of the synthesis column (i.e., the amount of 2,3-butanediol was below the detection limit). The third column (Example 1) summarises the performance of the synthesis column in case there was about 1.5 wt. % of 2,3-butanediol in the feed material. The performance of the lactide synthesis column is defined by the crude production rate and the amount of side reactions occurring. The operating conditions were kept as constant as possible.

The results demonstrate the addition of 2,3-butandiol, surprisingly, speeds up the lactide formation rate. The crude rate is higher when compared to the reference case at the same operating level, pressure, temperature and catalyst level. In addition, the formation of acetaldehyde and racemisation are suppressed.

| Operating conditions | | |
|---|---|---|
| lactide synthesis reactor | Reference | Example 1 |
| Level (%) | 25 | 25 |
| Pressure (mbar) | 3.4 | 4.4 |
| Catalyst concentration | 100% | 110% |
| (%, relative to reference) | | |
| Estimated weight ratio of | 1.0:>30 | 1.3:1.0 |
| 2,3-butanediol to dilactyl | | |
| ether units in the co-oligomers | | |
| 2,3-Butanediol in feed lactic | <0.03 | 1.5 |
| acid (wt %, after methanolysis) | | |

| Performance indicators | Reference | Example 1 |
|---|---|---|
| Net crude production rate (%) | 100% | 130% |
| Viscosity (%, | 100% | 21% |
| relative to reference) | | |
| Racemisation (%, | 100% | 71% |
| relative to reference) | | |
| Acetaldehyde production | 100% | 22% |
| (%, corrected for crude rate, | | |
| relative to reference) | | |

### Example B: Optimizing 2,3-Butanediol Concentration to Further Improve Lactide Synthesis Rates and Racemisation Rates

The example below compares four different cases. The first two cases (Reference and Example 1) have already been described in Example A.

In the third case (Example 2), the lactic acid used as feed material consisted of a blend of commercial lactic acid and recycled lactic acid. As shown in the Table below, the feed stream sent to the oligomerisation reactor in Example 2 contained 0.1 wt. % of 2,3-butanediol. The co-oligomers formed in the oligomerisation reactor were, thus, co-oligomers derived from lactic acid and from 0.1 wt. % of 2,3-butanediol.

In the fourth case (Example 3), the lactic acid used as feed material consisted of a blend of commercial lactic acid and recycled lactic acid. As shown in the Table below, the feed stream sent to the oligomerisation reactor in Example 3 contained approximately 0.4 wt. % of 2,3-butanediol (this particular value was estimated based on concentration factor observed in the lactide synthesis column). The co-oligomers formed in the oligomerisation reactor were, thus, co-oligomers derived from lactic acid and from approximately 0.4 wt. % of 2,3-butanediol.

The oligomers were fed to the same continuous lactide synthesis reactor as used in the other examples. The mixture was converted into crude lactide in presence of a Sn-based catalyst (760-1040 ppm Sn), at a temperature of 185 to 200 °C, and at a pressure of from 250 to 500 Pa. A heavies stream was continuously extracted from the bottom of the reactor and the composition of the heavies stream was analysed.

The fourth column in the table below (Example 2) shows the performance in the case where there was 0.1 wt. % of 2,3-butanediol in the co-oligomers. As in Example 1, the performance of the lactide synthesis column is defined by the crude production rate and the amount of side reactions occurring. The operating conditions were again kept as constant as possible.

When compared with the results of the reference example, the results of Example 2 show an improved net production rate, as well as a lower viscosity. In addition, the formation of acetaldehyde was reduced compared to the reference example.

The fifth column in the table below (Example 3) shows the performance in the case where there was about 0.4 wt. % of 2,3-butanediol in the co-oligomers. As in Example 1, the performance of the lactide synthesis column is defined by the crude production rate and the amount of side reactions occurring. The operating conditions were again kept as constant as possible.

When compared with the results of the reference example, Example 1, and Example 2, the results surprisingly demonstrate there is an optimal amount of 2,3-butandiol to be used in the co-oligomers for lactide synthesis. The crude rate was higher for the case where there was about 0.4 wt. % of 2,3-butanediol present in the co-oligomers, even though the synthesis column was operated at a lower temperature and with a lower catalyst concentration. In addition, racemisation was surprisingly suppressed further compared to Example 1 (and Example 2).

| Operating conditions | | Exam- | Exam- | Exam- |
|---|---|---|---|---|
| lactide synthesis reactor | Reference | ple 1 | ple 2 | ple 3 |
| Level (%) | 25 | 25 | 25 | 25 |
| Pressure (mbar) | 3.4 | 4.4 | 2.7 | 3.4 |
| Catalyst concentration | 100% | 110% | 56% | 79% |
| (%, relative to reference) | | | | |
| Estimated weight ratio of | 1:>30 | 1.3:1 | 1:2.9 | 1.0:1.0 |
| 2,3-butanediol to dilactyl | | | | |
| ether units in the oligomers | | | | |
| 2,3-Butanediol in feed | <0.03 | 1.5 | 0.1 | 0.4 |
| lactic acid (wt %) | | | | |

| | | Exam- | Exam- | Exam- |
|---|---|---|---|---|
| Performance indicators | Reference | ple 1 | ple 2 | ple 3 |
| Net crude production rate (%) | 100% | 130% | 156% | 188% |
| Viscosity (%, | 100% | 21% | 60% | 41% |
| relative to reference) | | | | |
| Racemisation (%, | 100% | 71% | 114% | 64% |
| relative to reference) | | | | |
| Acetaldehyde production | 100% | 22% | 23% | 27% |
| (%, corrected for crude rate, | | | | |
| relative to reference) | | | | |

1. A process for manufacturing a lactide-containing composition, the process comprising: providing co-oligomers derived from lactic acid and from 0.05 to 5.0 wt. % of 2,3-butanediol, calculated on the total weight of the co-oligomers as determined by gas chromatography (GC); and depolymerizing the co-oligomers to form a lactide-containing composition.

2. The process of claim 1, wherein the co-oligomers comprise from 0.30 to 1.3 wt. % of 2,3-butanediol, calculated on the total weight of the co-oligomers as determined by GC.

3. The process of claim 1, wherein the co-oligomers comprise from 95.0 to 99.95 wt. % lactic acid, calculated on the total weight of the co-oligomers as determined by GC.

4. The process of claim 1, wherein the co-oligomers further comprise dilactyl ether units, and wherein the 2,3-butanediol and the dilactyl ether units are present in a weight ratio of 5:1 to 1:5.

5. The process of claim 4, wherein the 2,3-butanediol and the dilactyl ether units are present in a weight ratio of about 1:1.

6. The process of claim 1, wherein the lactide-containing composition comprises from 60 to 99 wt. % L-lactide.

7. The process of claim 1, wherein providing the co-oligomers comprises oligomerizing lactic acid and 2,3-butanediol.

8. The process of claim 1, further comprising:
separating a mixture comprising lactic acid, lactide, and 2,3-butanediol in one or more steps to form a lactic acid-containing composition comprising lactic acid and 0.05 to 12.0 wt. % of 2,3-butanediol;
using at least a portion of the lactic acid-containing composition to provide the co-oligomers; and
depolymerizing the co-oligomers to form the lactide-containing composition.

9. The process of claim 8, wherein the lactic acid-containing composition is hydrolyzed and purified before being used to provide the co-oligomers.

10. The process of claim 1, further comprising purifying the lactide-containing composition by distillation, solvent crystallization, melt crystallization, or a combination thereof.

11. A process for producing a polymer, the process comprising:
manufacturing the lactide-containing composition by the process of claim 1; and
polymerizing at least a portion of the lactide-containing composition, optionally in combination with one or more other lactide-containing compositions, to form a polymer.

12. A composition comprising co-oligomers derived from lactic acid and from 0.05 to 5.0 wt. % of 2,3-butanediol, calculated on the total weight of the co-oligomers as determined by GC.

13. The composition of claim 12, wherein the co-oligomers comprise from 0.10 to 4.5 wt. % of 2,3-butanediol, calculated on the total weight of the co-oligomers as determined by GC.

14. The composition of claim 13, wherein the co-oligomers comprise from 0.30 to 1.3 wt. % of 2,3-butanediol, calculated on the total weight of the co-oligomers as determined by GC.

15. The composition of claim 12, wherein the co-oligomers further comprise dilactyl ether units, and wherein the 2,3-butanediol and the dilactyl ether units are present in a weight ratio of 5:1 to 1:5.

16. A process for manufacturing the composition of claim 12, the process comprising oligomerizing a composition comprising lactic acid and from 0.05 to 12 wt. % of 2,3-butanediol.
